(19) United States
(12) Patent Application Publication
(10) Pub. No.: US 2026/0236735 A1
(43) Pub. Date: Aug. 13, 2026

(54) Polymer Glass Transition Temperature Estimation Method and System Thereof

(71) Applicant: LG Chem, Ltd., Seoul (KR)

(72) Inventors: Hee Yeon Moon, Daejeon (KR); Jae Soon Bae, Daejeon (KR); Kuy Hwang Lee, Daejeon (KR); Hyeon Ah Shin, Daejeon (KR)

(73) Assignee: LG Chem, Ltd., Seoul (KR)

(21) Appl. No.: 18/998,800

(22) PCT Filed: Dec. 27, 2023
(86) PCT No.: PCT/KR2023/021741

§ 371 Date: Feb. 24, 2026

(30) Foreign Application Priority Data
Jan. 4, 2023 (KR) 10-2023-0001253

(51) Int. Cl. G06N 3/042; G06N 3/08

(52) U.S. Cl. CPC G06N 3/042; G06N 3/08

(57) ABSTRACT

A computer-implemented method for estimating a glass transition temperature of a polymer from a chemical structure of the polymer is provided. The method provides for graphically representing a chemical structure of a polymer, and a method and a system for calculating a glass transition temperature of a polymer from graph data of the polymer by machine-learning a chemical structure of the polymer based on information defining an inter-attachment relationship between respective atoms constituting a repeat unit of the polymer and an attaching node to which repeated repeat units are attached, in which, in addition to the chemical structure of the polymer, molecular weight information is reflected.

### CROSS-REFERENCE TO RELATED APPLICATIONS

The present application is a 371 National Stage entry of PCT Application No. PCT/KR2023/021741 filed on Dec. 27, 2023, and which claims priority to Korean Patent Application No. 10-2023-0001253 filed on Jan. 4, 2023, the disclosures of which are incorporated by reference herein.

### TECHNICAL FIELD

The present disclosure relates to a method and a system for estimating a glass transition temperature of a polymer using an artificial intelligence neural network.

### BACKGROUND

In order to input a structure of a compound into a computer device and perform predetermined processing, various methods for representing the structure of the compound as a computer device recognizable one have been suggested. Representative methods include a method using a simplified molecular-input line-entry system (SMILES) for representing the structure of a compound as a string or a method for representing the string converted with the SMILES as a descriptor.

However, it is difficult to represent polymers, which are not single molecules, in the SMILES method, and thus BigSMILES or Hierarchical Descriptor are suggested.

However, in such technologies in the prior art, there are problems in that the larger a molecule is, the more more complex the method for representing the molecule, a high molecule is represented only in fragments and thus there is no technology to process the high molecule based on the representation, properties of a polymer in which repeat units are infinitely repeated may not be properly represented, and so on.

Meanwhile, in estimating certain properties of a polymer, in the prior art, as presented in the non-patent documents below, by reflecting structural properties of the polymer, the certain properties of the polymer have been estimated from the reflected properties.

However, among the properties of a polymer, since the glass transition temperature (T_{g}) is affected not only by the structure of the polymer but also by the molecular weight of the polymerized polymer, there is a problem in that it is not possible to estimate the accurate glass transition temperature from the technologies in the prior art.

The technologies in the prior art are as follows:
Korean Patent Laid-Open Publication No. 2021-0042777 A
Korean Patent Laid-Open Publication No. 2021-0110539 A
Machine-learning predictions of polymer properties with Polymer Genome, Journal of Applied Physics 128, 171104, 2020
Machine learning discovery of high-temperature polymers, Matter, Volume 4, Issue 5, 5 May 2021, pages 1454-1456

### DISCLOSURE OF THE INVENTION

### Technical Problem

Accordingly, in order to solve the above-described problems, the present disclosure is intended to provide a method for providing a data structure representing a polymer material as graphic data, and furthermore, estimating a glass transition temperature of a polymer using the data structure representing the graphic data of the polymer, in which a molecular weight element of the polymer is reflected.

### Technical Solution

In order to solve the above problems, according to the present disclosure, there is provided a computer-implemented method for graphically describing a chemical structure of a selected polymer and calculating an estimated value of a glass transition temperature of the polymer, the computer-implemented method including a polymer glass transition temperature property information estimation artificial neural network acquisition step of generating a machine-trained graph artificial neural network to estimate glass transition temperature property information about the selected polymer based at least in part on chemical structure data related to the selected polymer, a polymer graph data acquisition step of acquiring polymer graph data describing a chemical structure of the selected polymer as graphic data; a polymer graph input step of providing the acquired polymer graph data as input to the graph neural network, a polymer glass transition temperature property information acquisition step of receiving the polymer glass transition temperature property information of the selected polymer from output of the graph neural network, and a polymer glass transition temperature calculation step of calculating a glass transition temperature of the selected polymer by substituting molecular weight information about the selected polymer into the polymer glass transition temperature property information.

In this case, the polymer graph data describing the chemical structure of the selected polymer as graph data may include at least one of polymer graph node attachment data that is data representing an inter-attachment relationship between nodes corresponding to respective atoms constituting a repeat unit for composing the polymer and attaching nodes that are attached to an attach point that is a point where the repeat units are repeatedly attached among the nodes, polymer node attribution information that is information representing attribute information about the nodes corresponding to the respective atoms constituting the repeat unit for composing the polymer and attributes of attaching nodes that are attached to the attach point that is a point where the repeat units are repeatedly attached among the nodes, and polymer edge attribute information that is information representing attributes of an edge for attaching the nodes corresponding to the respective atoms constituting the repeat unit for composing the polymer to each other and an attach edge for attaching at least one of the nodes to the attaching node.

According to the present disclosure, there is also provided a glass transition temperature calculation system for polymers, including a data input unit that receives polymer graph data describing a chemical structure of a selected polymer as graphic data and molecular weight data of the polymer, an artificial neural network model unit that estimates and calculates glass transition temperature property information about the polymer from the polymer graph data, and a glass transition temperature calculation unit that calculates a glass transition temperature of the polymer from the glass transition temperature property information about the polymer and the molecular weight data of the polymer, and in this case, the glass transition temperature property information about the polymer is T_{g}, inf value and K in the equation below, the molecular weight data of the polymer is Mₙ in the equation below, and the glass transition temperature calculation unit calculates a glass transition temperature T_{g} from the equation below.

$${T}_{g} = {T}_{g , inf} - \frac{K}{{M}_{n}}$$

(T_{g} is a glass transition temperature of a polymer with a molecular weight Mₙ, T_{g,inf} is a glass transition temperature when the molecular weight is infinite, K is a variable related to a free volume of the polymer, and Mₙ is a number average molecular weight of the polymer).

### Advantageous Effects

According to the present disclosure, by providing a data structure that can represent a polymer in computer-usable graphical data, it is possible to implement a method and a system for estimating property information about a polymer material by machine-learning a relationship between a molecular structure and property information about the polymer material through a graph neural network (GNN) and a message passing neural network (MPNN). Furthermore, by reflecting molecular weight factors in estimating a glass transition temperature of a polymer, it is possible to provide a method and a system for estimating a glass transition temperature of a polymer with improved accuracy as compared with the prior art.

### BRIEF DESCRIPTION OF THE DRAWINGS

The following drawings attached to the specification illustrate preferred aspects of the present disclosure by example, and serve to enable technical aspects of the present disclosure to be further understood together with detailed description of the disclosure given above, and therefore the present disclosure should not be interpreted only with matters in such drawings.

FIG. 1 is a diagram illustrating graphical representations of monomers and polymers according to the prior art.

FIG. 2 is a diagram illustrating graphical representations of a polymer according to the present disclosure.

FIG. 3 is an exemplary diagram for describing the graphical representation of the polymer according to the present disclosure.

FIG. 4 is a diagram illustrating a procedure for implementing an artificial neural network for calculating a glass transition temperature of a polymer using a graphical representation of a polymer according to the present disclosure.

FIG. 5 is a block diagram of a system for estimating a glass transition temperature of a polymer according to the present disclosure.

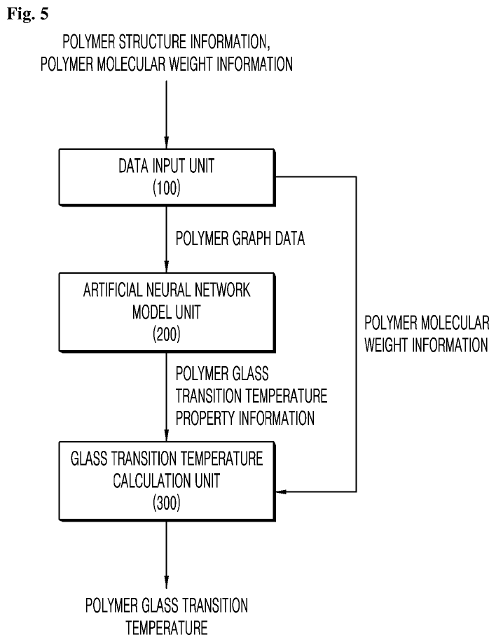

### DETAILED DESCRIPTION

Names of respective reference characters used in the present invention are as follows:
10 Node
20 Edge
30 Attach point
40 Attach edge
100 Data input unit
200 Artificial neural network model unit
300 Glass transition temperature calculation unit

### 1. Graph Representation Method for Polymer According to the Present Disclosure

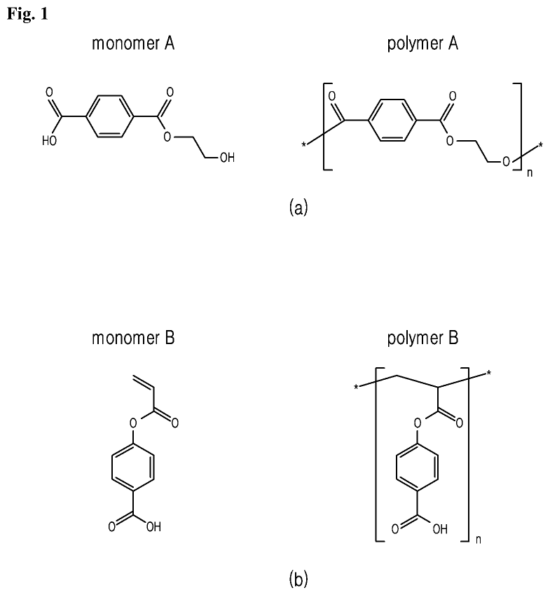

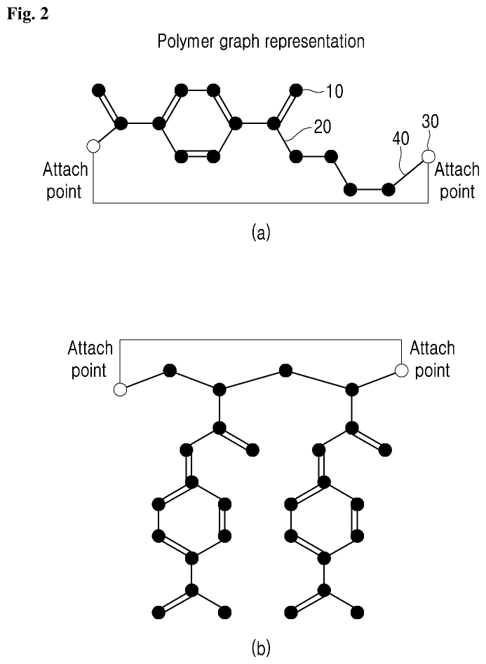

First, a method of graphically representing polymers according to the present disclosure will be described with reference to FIGS. 1 and 2.

(a) and (b) in FIG. 1 are diagrams illustrating structures of different polymers and the monomers composing each polymer. However, in trying to process molecular structure data using an artificial neural network, especially in representing a molecular structure as a graph for application to a GNN or the like, there is no method of effectively representing a chemical structure of a polymer as a graph.

Monomers that compose polymers illustrated in (a) and (b) in FIG. 1 are different from each other, but since they are the same except for some of the monomer terminals, graphs of the monomers are represented very similarly, and thus it is inaccurate to represent graphs of the polymers simply as graphs of the monomers and to represent the graphs of the polymers as repetitions of the graphs of the monomers.

Therefore, the present disclosure suggests a new method of representing a polymer graph as illustrated in FIG. 2. Hereinafter, a polymer graph data method according to the present disclosure will be described with reference to FIG. 2.

First, in a polymer graph representation method of the present disclosure, monomers for composing a polymer and a repeat unit that is repeated to compose the polymer are derived. FIG. 1 illustrates an example of polymer representation in the prior art and a representation of the monomers for composing the polymer, and FIG. 2 is a diagram illustrating the repeat units for composing the polymer of FIG. 1. In the repeat unit of FIG. 2, for the polymer representation method of the present disclosure, each atom is expressed as a node 10, and coupling between the atoms is expressed as an edge 20. Next, when the repeat unit is repeated to compose the polymer, which is one of the features of the present disclosure, a point where the repeat units are repeatedly attached is expressed as an attach point 30, and a node to which the attach point is attached is expressed an “attaching node.” In addition, the attach point 30 is a virtual representation of the attaching nodes of neighboring repeat units attached by repetition, and the attachment for attaching the attach point to the attaching node is expressed as an attach edge 40.

According to the polymer graph representation method of the present disclosure as described above, when an artificial neural network model is trained based on chemical structures of polymers, in particular, when learning is performed by applying an artificial neural network model such as a message passing neural network (MPNN) and a graph neural network (GNN) based on message passing, even by adding a minimal amount of information in addition to monomer structure information, learning to be performed by transferring information about an opposite node where a monomer is attached is possible and learning to be performed by reflecting the unit repetition characteristics of the polymer is possible.

### 2. Computer-Usable Datafication of Graph Representation of Polymer According to the Present Disclosure

The present disclosure converts the chemical structure of a polymer into a data structure capable of being used in computer processing, that is, “graph data” according to the graph representation method that graphically represents the above-mentioned molecular or chemical structure of the polymer. In the present disclosure, this is referred to as “polymer graph data”.

According to the present disclosure, polymer graph data that describes a chemical structure of a given or selected polymer in graphic data may include at least one of node data that is data of nodes 10 representing two or more nodes representing respective atoms constituting a repeat unit of a monomer for composing the polymer and data related to the nodes 10, edge data that is data related to one or more edges 20 representing coupling between respective nodes, attaching node data that is data related to at least one attaching node that is a point where repeat units for composing the polymer are repeatedly attached among the nodes, and attach edge data that is data related to the attach edge 40 attaching the attach point 30 to the attaching node.

A difference between the monomer graph data and the polymer graph data of the present disclosure is that the polymer graph data of the present disclosure represents the node 10 attached to the attach point 30 and its edge information as the attaching node and the attach edge in the graph data representation of the repeat unit including the monomer graph data.

### 2.1. Adjacency Matrix and Feature Matrix

When converting the graph structure of the monomer into data capable of being used on a computer, there may be various data representation methods, but typically, edge data and node data may be represented as an adjacency matrix and a feature matrix, respectively. The edge data is data related to coupling between atoms, and the node data is data related to each atom.

According to a polymer representation method according to the present disclosure, in addition to a monomer graph representation method in the prior art, the polymer graph is able to be converted into computer-recognizable data by additionally adding data for the nodes and the attach edge 40 attached to the attach point 30.

### (1) Adjacency Matrix Expression of Monomer Graph

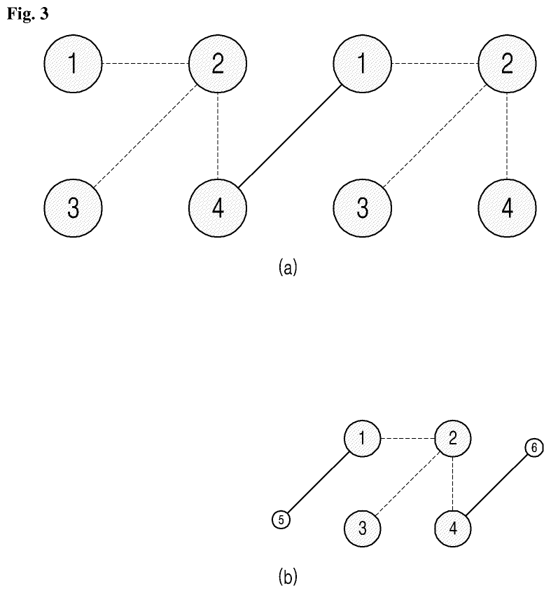

For the sake of simplicity of description, when a simple structure, as illustrated in FIG. 3, is described by way of example, an adjacency matrix A of a repeat unit of a monomer consisting of nodes 1, 2, 3, and 4 may be represented as follows.

For the sake of simplicity of description, when a simple structure, as illustrated in FIG. 3, is described by way of example, an adjacency matrix A of a repeat unit of a monomer consisting of nodes 1, 2, 3, and 4 may be represented as follows.

$$Monomer adjacency matrix A = [ \begin{matrix}0 & 1 & 0 & 0 \\ 1 & 0 & 1 & 1 \\ 0 & 1 & 0 & 0 \\ 0 & 1 & 0 & 0\end{matrix} ]$$

The adjacency matrix A has as many rows and columns as the number of nodes, and each element A_{i,j} is data indicating whether an i (i=1, . . . , 4)-th node and a j (j=1, . . . , 4)-th node are attached to each other. A value of each matrix element represents data on attachment to other nodes. For example, since a node 1 is only attached to a node 2 and not to nodes 1, 3, and 4, A_{i, j} (j=1, . . . , 4) of the adjacency matrix=[0 1 0 0] is represented.

### (2) Adjacency Matrix Expression of Polymer Graph According to the Present Disclosure

According to the polymer graph data representation method of the present disclosure, a polymer adjacency matrix PA, which is an adjacency matrix representation of the polymer graph according to the example of FIG. 3, may be expressed as follows:

$$Polymer adjacency matrix PA = [ \begin{matrix}0 & 1 & 0 & 1 \\ 1 & 0 & 1 & 1 \\ 0 & 1 & 0 & 0 \\ 1 & 1 & 0 & 0\end{matrix} ]$$

As illustrated in FIG. 3, the polymer adjacency matrix PA has two attach points (5, 6) added, and these points refer to nodes 4 and 1 of an adjacent monomer, respectively, and thus the nodes 1 and 4 are represented as being attached to nodes 4 and 1, respectively. That is, since there is no node attached to nodes 1 and 4 in the monomer, matrix values indicating attachment relationships of the node 1 and the node 4 are A_{1,4}=0, A_{4, 1}=0, but in the polymer representation method of the present disclosure, since the nodes 1 and 4 are attached to nodes 4 and 1 of adjacent monomers, respectively, the polymer adjacency matrix PA is expressed as PA_{1,4}=1, PA_{4,1}=1, and contains repetitive attach data of the polymer. That is, since the nodes 1 and 4, which have not been attached in the monomer, are attached to each other in the polymer, the nodes 1 and 4 are represented as being attached to each other in the polymer adjacency matrix that displays the attach edge data.

However, in this case, since the nodes 1 and 4 are not attached to each other within the monomer, in order to indicate that the attachment is not attachment within the monomer, but attachment to the node of the adjacent repeat unit, a distinction may be made by expressing the node attachment relationship as a negative number to express the polymer adjacency matrix as follows, giving a feature of “attaching node attached to the attach point” to the feature of the corresponding node in a node feature matrix to be described below, or giving an adjacent repeat unit attachment attribute to an edge feature matrix.

$$Polymer adjacency matrix {PA}^{′} = [ \begin{matrix}0 & 1 & 0 & - 1 \\ 1 & 0 & 1 & 1 \\ 0 & 1 & 0 & 0 \\ - 1 & 1 & 0 & 0\end{matrix} ]$$

In other words, the graph data that represents the chemical structure of the polymer as a graph and describes it as graphic data according to the present disclosure includes, in addition to the graph data of the repeat unit of the monomer, attaching node data representing an attaching node that is a node to which the monomers are repeatedly attached and data of the attach edge 40 that is attached to the attaching node.

The expression method of the polymer adjacency matrix PA indicates that the nodes 1 and 4 of the repeat unit of the monomer consisting of nodes 1, 2, 3, and 4 are attached to the nodes 4 and 1 of the neighboring repeat unit, respectively, and this attachment is repeated. However, at this time, the nodes 1 and 4 in FIG. 3 are not attached to each other within the monomer, but are attached to the nodes of the adjacent repeat unit, so that these nodes may be distinguished by designating the nodes as “attaching nodes” and giving the attaching node attribute to the node feature matrix or giving the adjacent repeat unit attachment attribute to the edge feature matrix.

This may be used as an embedding procedure to convert the chemical structure of a polymer into data capable of being processed by a computer, which is achieved by a polymer edge variable setting step of setting an inter-attachment relationship between nodes representing respective atoms constituting the monomer repeat unit of the selected polymer and the attaching nodes that are points where the monomers are repeatedly attached as a polymer attachment edge variable, and a polymer graph edge data assignment step of assigning an attribute value of the inter-attachment relationship between the nodes and the attaching nodes to the set polymer edge variable. As seen in the polymer adjacency matrix PA above, the attribute value of the inter-attachment relationship between the nodes and the attaching nodes may be determined as “1” and “0”, indicating attachment/non-attachment, the matrix expression of which is the polymer adjacency matrix. The polymer adjacency matrix of the present disclosure has the same component values as a typical adjacency matrix, but as described above, it differs from the typical adjacency matrix by adding attaching nodes to a typical single molecule graph node.

### (3) Feature Matrix of Monomer Graph

In addition to the adjacency matrix showing the attachment relationship between nodes, the graph representation of a compound may have a feature matrix representing predetermined attribute information about each node and predetermined attribute information about each edge as graph data. In an exemplary monomer graph of FIG. 3, a monomer node feature matrix containing attribute value information (e.g., three pieces of attribute information) about each atom can be expressed as follows:

$$Monomer node feature matrix NF = [ \begin{matrix}0 & 1 & 0 \\ 1 & 0 & 1 \\ 0 & 1 & 0 \\ 0 & 1 & 0\end{matrix} ]$$

A node feature matrix NF may have as many rows as the number of nodes and as many columns as the number of types of feature values to be expressed for each node, and thus may express a certain attribute to be expressed for each atom. Each component NF_{i,j} has a j-th feature value of an i-th node. For the purpose of description, the monomer feature matrix NF is described as an example in which each node has three arbitrary attribute values.

Information of the monomer graph may be represented as an edge feature matrixEF, where each row may represent each edge of a monomer and column data of each row may be represented to express a predetermined attribute value of each edge. Edge attribute values may include the type of chemical bond (single bond, double bond, or the like), a ring status, a conjugation status, or the like. For example, in the case of the monomer in FIG. 3, since there are three attach edges, the edge feature matrix has three rows, and edge attribute values intended to contain information are assigned to respective columns. An example of the edge feature matrix with three random edge attribute values is as follows.

$$Monomer edge feature matrix EF = [ \begin{matrix}1 & 0 & 0 \\ 0 & 1 & 1 \\ 0 & 0 & 0\end{matrix} ]$$

### (4) Feature Matrix of Polymer Graph According to the Present Disclosure

Polymer graph data according to the present disclosure may have separate attribute values added to the attribute values of the monomer node feature matrix described above. For example, a polymer node feature matrix PNF, which is a feature matrix of the exemplary polymer graph in FIG. 3, may also have three columns plus attribute data, as in the example illustrated below, and express the added column as attribute values indicating whether respective nodes are an attaching node. For example, the polymer node feature matrix PNF below may have attaching node attribute information in a fourth column, as illustrated below, in addition to the monomer node feature matrix NF containing three pieces of attribute information about the previous four nodes. In the example of FIG. 3, since the nodes 1 and 4 are attaching nodes attached to adjacent repeat units, the nodes 1 and 4 are represented as having a value of “1”, and other nodes are represented as having a value of “0” in the fourth column.

$$Polymer node feature matrix PNF = [ \begin{matrix}0 & 1 & 0 & 1 \\ 1 & 0 & 1 & 0 \\ 0 & 1 & 0 & 0 \\ 0 & 1 & 0 & 1\end{matrix} ]$$

This may be achieved by a polymer node variable setting step of setting each of atoms of a monomer for composing a selected polymer and an attaching node that is a point to which the monomers are repeatedly attached as polymer node variables and a polymer graph node data assignment step of assigning an attaching node to be attached to the set polymer node variable and assigning a node attribute value to each of the nodes. The node attribute values assigned to nodes may include atomic numbers, hybridizations (SP3, SP2, or the like), the number of hydrogens, the number of electrons, ring status, or the like, and in particular, as one embodiment of the present disclosure, may have an attaching node attribute value, which is an attribute value of whether a node is attached to an adjacent repeat unit.

As another embodiment of the present disclosure, attaching node data may be represented by an edge feature matrix. For example, like the polymer node feature matrix described above, instead of expressing with a node attribute value whether a node is an attaching node, or together with the node attribute value, an attribute value expressing whether a node is an attaching node is expressed in the edge feature matrix. In the example below representing (b) in FIG. 3, it can be seen that data in fourth and fifth rows are added to contain information on the edges attached to the attaching nodes 1 and 4, that is, edges 5_1 and 4_6 in (b) in FIG. 3 and an attribute information column expressing whether a node is an attaching node is added in the fourth column.

$$Polymer edge feature matrix PEF = [ \begin{matrix}1 & 0 & 0 & 0 \\ 0 & 1 & 1 & 0 \\ 0 & 0 & 0 & 0 \\ 0 & 0 & 0 & 1 \\ 0 & 0 & 0 & 1\end{matrix} ]$$

That is, according to the present disclosure, the method of representing the structure of a polymer as a graph and converting it into computer-usable data may be selected from one or more of the methods below.
\textcircled{1} In the adjacency matrix expressing the attachment relationship between respective nodes of a repeat unit formed by at least one monomer, a component of an “attaching node” attached to an adjacent repeat unit is assigned as an “attachment” attribute.
\textcircled{2} An “attachment status” attribute field is given to a node attribute matrix expressing attributes of respective nodes of the repeat unit, and “attachment” is assigned to the “attachment status” attribute field of the “attaching node”.
\textcircled{3} Edges attached to the attaching node are added as attach edges to the edge attribute matrix of the repeat unit, “attachment status” attribute fields are given to the edge attributes, and “attachment” is assigned to the corresponding fields of the attach edges.
   2.2. Method of Converting Chemical Structure of Polymer into Data Capable of being Processed by Computer

In the present disclosure, in training the artificial neural network model to be described below and estimating a property of a polymer material using the trained artificial neural network, the chemical structure of the polymer material has to be converted into data so that the polymer may be processed by a computer, and the conversion into data may be performed as follows according to a method of the present disclosure.

### (1) Repeat Unit Information Acquisition Step

This is a step of acquiring information on the repeat unit, which is a unit for a polymer composed by repeating a monomer. In order to convert attaching node data in the repeat unit into data, which is one of the features of the present disclosure, the information on the repeat unit for composing the polymer is acquired. This is a step to confirm whether the repeat unit repeated to compose the polymer is repeated as a single monomer or as a combination of single monomers, and to describe the repeat unit in a graphical representation. The repeat unit may be a single monomer as shown in (a) in FIG. 2 or may be a case where monomers are attached to each other as shown in (b) in FIG. 2.

### (2) Polymer Graph Node Attachment Information Assignment Step

In the present disclosure, a method for processing polymer graph data includes a step of setting, as a variable, an inter-attachment relationship between nodes representing respective atoms constituting a repeat unit of a polymer selected as an analysis target and attaching nodes attached to an attach point that is a point where the repeat units are repeatedly attached among the nodes.

An attribute value of the inter-attachment relationship between the nodes and attaching nodes is assigned to the polymer node attachment variable set in this way. To assign the attribute value of each attachment relationship to the polymer node attachment variable, the polymer adjacency matrix mentioned above may be utilized.

### (3) Polymer Graph Node Attribute Information Assignment Step

The method for processing polymer graph data of the present disclosure may also include a polymer node attribute variable setting step of setting, as polymer node attribute variables, attribute information about nodes corresponding to respective atoms constituting the repeat unit for composing the selected polymer and attribute information about the attaching nodes attached to the attach point that is a point where the repeat units are repeatedly attached among the nodes.

The method additionally includes a polymer graph node attribute information allocation step of assigning attribute values of the nodes and attaching nodes to the polymer node attribute variables set in this way. The node attribute information has an attribute value indicating whether the node is an attaching node or not, and as node attribute information for the attaching node, unlike nodes that are not the attaching node, an attaching attribute value indicating that the node is attached to the attaching node of neighboring repeat unit within the polymer is given.

### (4) Polymer Graph Edge Attribute Information Assignment Step

The method for processing polymer graph data of the present disclosure may also include a polymer edge attribute variable setting step of setting, as polymer edge attribute variables, information about edges inter-attaching nodes corresponding to respective atoms constituting the repeat unit for composing the selected polymer and an attach edge attaching at least one among the nodes to the attaching node. The attach edge is an edge attaching the attaching node to another node.

The present disclosure also includes a polymer graph edge attribute information assignment step of assigning attribute values of the edges and the attach edges to the set polymer edge attribute variables, and the attach edge is assigned an attach edge attribute value, which means that the edge attaches the attaching node to another node, as its edge attribute variable.

### 3. Method for Estimating Glass Transition Temperature of Polymer Using Graph Representation of Polymer According to the Present Disclosure

Hereinafter, a method for processing polymer data in an artificial intelligence neural network according to the polymer graph representation method of the present disclosure described above will be described.

The present disclosure provides a method for constructing an artificial neural network model that estimates a glass transition temperature of a polymer by applying the above-described polymer graph representation method and estimating the glass transition temperature of the polymer through machine learning.

### 3.1. Polymer Glass Transition Temperature Estimation Model Generation Method

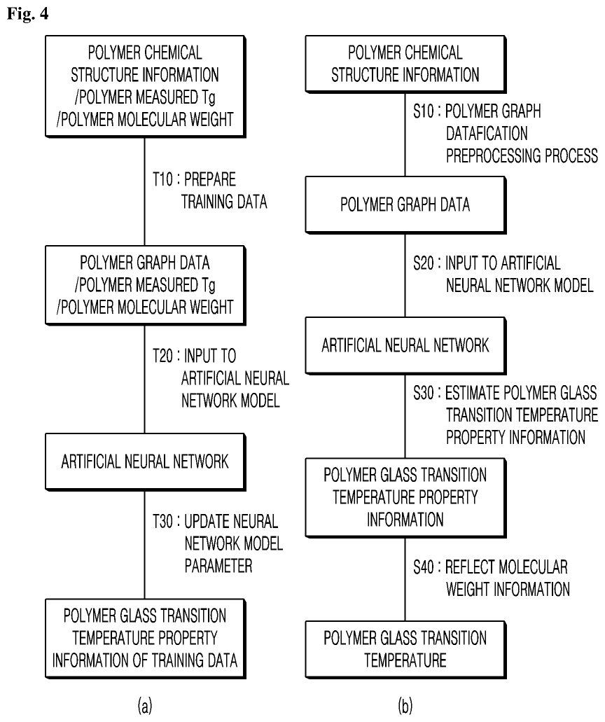

(a) in FIG. 4 illustrates a polymer glass transition temperature estimation model generation method according to the present disclosure. Based on this, a computer-implemented polymer glass transition temperature estimation model generation method for estimating a glass transition temperature of a selected polymer from a chemical structure of the corresponding polymer according to the graph data representation method of the present disclosure will be described.

The polymer glass transition temperature estimation model of the present disclosure is configured to estimate the glass transition temperature by applying the relationship between the glass transition temperature and molecular weight of a polymer defined by the Flory-Fox Equation of [Equation 1]. The glass transition temperature T_{g} of a polymer is a physical property value that is affected by both the molecular structure and the molecular weight, but the technology in the prior art fails to reflect the effects of both the molecular structure and the molecular weight in estimating the glass transition temperature. The present disclosure has implemented an estimation model with improved accuracy by reflecting both the molecular structure and the molecular weight in estimating the glass transition temperature using the equation below. In addition, it has become possible to estimate T_{g} according to changes in molecular weight for polymers of the same molecular structure.

$${T}_{g} = {T}_{g , inf} - \frac{K}{{M}_{n}} \qquad [ Equation 1 ]$$

T_{g,inf} is a glass transition temperature when the molecular weight is theoretically infinite, K is an experimental parameter related to a free volume in the polymer, and Mₙ is a number average molecular weight of the polymer.

Accordingly, the estimation model of the present disclosure includes an artificial neural network that estimates T_{g,inf} and K in Equation 1 from the molecular structure of a selected polymer, and is configured to calculate the glass transition temperature T_{g} of the selected polymer by applying the T_{g,inf} and K values output by the artificial neural network and the molecular weight of the selected polymer to Equation 1. In this estimation model, the following procedure is performed to generate the artificial neural network model that estimates T_{g,inf} and K values, which are glass transition temperature property information about the polymer.

### (1) Training Data Preparation Step (T10)

In order to generate the polymer glass transition temperature estimation model described above, training data for training the artificial neural network is prepared. The training data of the present disclosure contains a data set of {polymer graph data containing structure information about a sample polymer, a measured T_{g} value of the sample polymer, and a molecular weight Mₙ of the sample polymer}.

In addition, the training data of the present disclosure includes a predetermined number of data sets of {polymer graph data containing structure information about the sample polymer, the measured T_{g} value of the sample polymer, and the molecular weight Mₙ of the sample polymer} or more as a data set of a polymer with a similar structure but different molecular weight and a data set of a polymer with a different structure but the same molecular weight. By building the training data set in this way, the artificial neural network of the present disclosure, which will be described below, may be trained so that the estimated T_{g} value (T_{g} estimated value) reflects both the structural features and molecular weight features of the polymer.

### (2) Artificial Neural Network Model Construction and Learning Data Input Step (T20)

Next, the artificial neural network model to be trained using the training data is constructed. The artificial neural network in the present disclosure uses known artificial neural networks, and in particular, artificial neural networks such as a message passing neural network (MPNN) and a graph neural network (GNN) based on message passing may be applied.

For example, when the GNN is applied as the artificial neural network, describing a case of supervised learning, polymer training data containing data sets of {polymer graph data containing structure information about the sample polymer, the measured T_{g} value of the sample polymer, and the molecular weight Mₙ of the sample polymer} built above is input into the GNN.

In this case, polymer graph data is input as an input value of the artificial neural network, and the known measured T_{g} value and molecular weight Mₙ of the corresponding polymer are input as true values (labeled data) of the output value of the neural network. In this case, the input polymer graph data may include at least one of the polymer graph node attachment information, the polymer graph node attribute information, and the polymer graph edge attribute information, which have been described above, and may be input by being embedded in the polymer adjacency matrix and polymer node/edge feature matrix.

### (3) Artificial Neural Network Training Step (T30)

This is a process of training the artificial neural network by updating neural network parameters of the artificial neural network based on the input polymer graph data, the molecular weight information, and glass transition temperature property information about the polymer. The learning process of the artificial neural network follows the known artificial neural network learning process, and as one example, the T_{g} estimated value is calculated by receiving polymer graph data of the training data and reflecting measured molecular weight information in the training data sets to an estimated value of the glass transition temperature property information about the polymer that is calculated from the artificial neural network according to the [Equation 1], and the neural network parameters of the artificial neural network are updated so that an error function defined as a difference between the T_{g} estimated value and the measured T_{g} value of the corresponding polymer in the training data is minimized. Through updating of the neural network parameters, the artificial neural network that calculates glass transition temperature property information from the structure of the polymer is completed.

As such, in the present disclosure, accurate T_{g} estimation in the polymer is possible because the artificial neural network is trained to minimize the error between the measured value and the T_{g} estimated value reflecting molecular weight information.

Meanwhile, in the present disclosure, the term “neural network parameter” of the artificial neural network contains weights and biases that are adjusted while the artificial neural network model is trained from training data, and such a parameter is adjusted so that the neural network model estimates a more accurate output from input data, and is used in the general sense of the term in the field to indicate that it is optimized to estimate optimal output during a machine learning process.

### 3.2. Polymer Glass Transition Temperature Estimation Method

A procedure will be described for estimating a glass transition temperature of a selected polymer by inputting graph data of the selected polymer into the artificial neural network generated according to the polymer glass transition temperature model generation method of the present disclosure and reflecting the number average molecular weight Mₙ of the selected polymer.

### (1) Polymer Graph Datafication Preprocessing Process (S10)

As illustrated in (b) FIG. 4, a preprocessing process for acquiring polymer graph data from the chemical structure of the selected polymer whose glass transition temperature is to be estimated is performed. As described above, the polymer graph data includes data on attaching nodes and the attach edges 40 between repeating monomers, in addition to node and edge data of the monomer for composing the polymer. That is, the polymer graph data is preprocessed to include at least one of the above-described polymer graph node attachment information, polymer graph node attribute information, and polymer graph edge attribute information.

### (2) Polymer Graph Data Input Step (S20)

This is the step of inputting the polymer graph data based on the chemical structure of the selected polymer into the trained learned neural network model. In this case, the input polymer graph data may include at least one of the polymer graph node attachment information, polymer graph node attribute information, and polymer graph edge attribute information, and may be input by being embedded in the polymer adjacency matrix and polymer node/edge feature matrix described above.

### (3) Polymer Glass Transition Temperature Property Information Estimation Step (S30)

This is a step in which the trained artificial neural network model calculates and outputs the glass transition temperature property information T_{g}, inf and K values of the selected polymer from the input polymer graph data of the selected polymer.

### (4) Molecular Weight Information Reflection Step (S40)

This is a step of calculating the glass transition temperature T_{g} using the above [Equation 1] from the glass transition temperature property information T_{g}, inf and K values calculated by the artificial neural network model. The molecular weight of the polymer is acquired and reflected by a known method.

### 4. a System for Calculating a Glass Transition Temperature of a Polymer Using a Graph Representation of the Polymer According to the Present Disclosure

### (1) Data Input Unit 100

A data input unit 100 is configured to receive polymer graph data that describes a chemical structure of a selected polymer as graphic data and molecular weight data of the polymer, and further includes a data preprocessing unit (not illustrated) for converting the chemical structure of the polymer into the polymer graph data and inputting the converted data into the data input unit. Molecular weight data (molecular weight information) may be acquired by known methods.

In inputting the polymer graph data into an artificial neural network model unit, the data preprocessing unit may generate and input a polymer adjacency matrix expressing the inter-attachment relationship between two or more nodes representing respective atoms constituting the single molecule and attachment relationship information between the attaching node and the nodes and a polymer edge attribute matrix including attribute information about one or more edges representing coupling between the respective nodes and an attach edge representing attachment between the nodes and the attaching node.

In this case, as described above, the polymer graph data may include at least one of polymer graph node attachment data that is data representing an inter-attachment relationship between nodes corresponding to respective atoms constituting a repeat unit for composing the polymer and attaching nodes that are attached to an attach point that is a point where the repeat units are repeatedly attached among the nodes, polymer node attribution information that is data representing attribute information about the nodes corresponding to the respective atoms constituting the repeat unit for composing the polymer and attributes of attaching nodes that are attached to the attach point that is a point where the repeat units are repeatedly attached among the nodes, and polymer edge attribute information that is information representing attributes of an edge for attaching the nodes corresponding to the respective atoms constituting the repeat unit for composing the polymer to each other and an attach edge for attaching at least one of the nodes to the attaching node.

### (2) Artificial Neural Network Model Unit 200

This is a configuration for estimating and calculating glass transition temperature property information about the polymer from the polymer graph data. The glass transition temperature property information about the polymer corresponds to T_{g,inf} and K in Equation 1 described above.

Such an artificial neural network model unit is configured to receive polymer graph data and output an estimated value of the glass transition temperature property information about the corresponding polymer, and includes a polymer glass transition temperature property information estimation artificial neural network that is machine-trained by calculating an estimated value of glass transition temperature property information about a predetermined learning polymer from polymer graph data of the learning polymer, calculating an estimated T_{g} value from the calculated estimated value of the glass transition temperature property information about the learning polymer and the measured molecular weight information about the learning polymer, and updating its neural network parameters so that an error function defined as a difference between the estimated T_{g} value and the measured T_{g} value of the polymer in training data of the training data is minimized.

### (3) Glass Transition Temperature Calculation Unit 300

A glass transition temperature calculation unit is configured to calculate the glass transition temperature of the polymer according to [Equation 1] described above from the polymer glass transition temperature property information calculated by the artificial neural network model unit and the molecular weight data of the polymer input from the data input unit.

The graph data representation method for graphically representing the structure of a polymer according to the present disclosure, and the method and the system for finally calculating the accurate glass transition temperature of the polymer by estimating the glass transition temperature property information about the polymer from graph data of the polymer and reflecting the molecular weight information of the polymer in the estimated glass transition temperature property information about the polymer have been described above.

With the method and the system for calculating the glass transition temperature of the polymer, which reflects the molecular weight information, according to the present disclosure, by separately considering the effect of molecular weight, estimation adjustment according to the molecular weight may be possible, which has the effect of making it possible to build a model capable of accurate estimation.

For example, when actual T_{g} of each of samples of three types of polymers A, B, and C with similar structures and molecular weights of 2,000, 50,000, and 80,000, respectively, is similar, according to a model in the prior art, since only the structures of molecules are reflected without considering the molecular weights, learning and estimation are performed on samples with similar structures so that the estimated T_{g} values are similar regardless of molecular weights.

That is, when the actual T_{g} (glass transition temperature) of polymer A with molecular weight K is R_T_{g}_A(K) and the estimated T_{g} thereof is P_T_{g}_A(K), for R_T_{g}_A (2,000)=R_T_{g}_B (50,000)≈R_T_{g}_C (80,000), the prior art trains a neural network and performs estimation to be P_T_{g}_A (2,000)≈P_T_{g}_B (50,000)≈P_T_{g}_C (80,000). In the case of the prior art, since the molecular weights are not reflected, even when the samples A, B, and C have the same molecular weight of 50,000, the prior art will estimate as P_T_{g}_A (50,000)≈P_T_{g}_B (50,000)≈P_T_{g}_C (50,000). However, when comparison is made with the same molecular weights for A, B, and C, which are R_T_{g}_A (2,000)≈R_T_{g}_B (50,000)≈R_T_{g}_C (80,000), according to Equation 1, in fact, R_T_{g}_A (50,000)>R_T_{g}_B (50,000)>R_T_{g}_C (50,000), and thus it is not possible to estimate the accurate glass transition temperature using the technology in the prior art.

In contrast, in the present invention, in the case of R_T_{g}_A (2,000)≈R T_{g}_B (50,000)≈R_T_{g}_C (80,000), even when the neural network has be trained to be P_T_{g}_A (2,000)≈P_T_{g}_B (50,000)≈P_T_{g}_C (80,000), since the training of the neural network is performed by reflecting the molecular weights, neural network parameters for adjusting the effect of molecular structure are learned differently, and thus for the same molecular weight, the estimation is made as P_T_{g}_A (50,000)>P_T_{g}_B (50,000)>P_T_{g} C (50,000).

In another case, when the molecular weights of polymers A, B, and C of similar structures are different and the actual T_{g} s are different, that is, when R_T_{g}_A (2,000)<R_T_{g}_B (50,000)<R_T_{g}_C (80,000), according to the prior art, since training is performed according to the molecular structures and the actual T_{g} S, which are label values, are given differently, it is learned and estimated that P_T_{g}_A (2,000)<P_T_{g}_B (50,000)<P_T_{g}_C (80,000) even when the polymers have similar structures. However, at this time, when the molecular weights of the polymers A, B, and C are the same, the actual T_{g} may be similar according to [Equation 1]. In this case, the estimated values should be derived as P_T_{g}_A (50,000)≈P_T_{g}_B (50,000)≈P_T_{g}_C (50,000), but since the model according to the prior art is trained based only on the molecular structures for the polymers A, B, and C with the same molecular weight, a problem occurs where it is estimated that P_T_{g}_A (50,000)<P_T_{g}_B (50,000)<P_T_{g}_C (50,000).

In contrast, according to the present invention, when R_T_{g}_A (2,000)<R_T_{g}_B (50,000)<R_T_{g}_C (80,000), since the training is performed by reflecting molecular weights even when training is performed so that P_T_{g}_A (2,000)<P_T_{g}_B (50,000)<P_T_{g}_C (80,000), for the same molecular weight, it is not estimated that P_T_{g}_A (50,000)<P_T_{g}_B (50,000)<P_T_{g}_C (50,000), but is estimated that P_T_{g}_A (50,000)≈P_T_{g}_B (50,000)≈P_T_{g}_C (50,000).

As described above, according to the present invention, since the neural network model is trained to estimate the glass transition temperature by reflecting not only the molecular structure of the polymer but also the molecular weight information about the polymer, the effect of each of molecular structure information and molecular weight information may be learned, and thus the glass transition temperature may be more accurately estimated than the prior art.

1. A method for generating a computer-implemented model that estimates a glass transition temperature of a selected polymer, the method comprising:
building a training data set with structure information of a plurality of learning polymers, known values of glass transition temperatures of the plurality of learning polymers, and measured molecular weights of the plurality of learning polymers;
constructing a polymer glass transition temperature property information estimation artificial neural network to receive polymer structure information and output an estimated value of the glass transition temperature property information thereof;
the training data set into the polymer glass transition temperature property information estimation artificial neural network; and
updating neural network parameters of the polymer glass transition temperature property information estimation artificial neural network based on a comparison result of an estimated value of the glass transition temperature calculated by reflecting the measured molecular weights of the corresponding polymers in the training data set in an estimated value of the glass transition temperature property information about the learning polymer output by the polymer glass transition temperature property information estimation artificial neural network and a measured value of the glass transition temperature of the learning polymer.

2. The method of claim 1, wherein the glass transition temperature property information is a T_{g,inf} value and a K value in Equation 1:
$${T}_{g} = {T}_{g , inf} - \frac{K}{{M}_{n}} , \qquad [ Equation 1 ]$$
wherein T_{g} is a glass transition temperature of a polymer with a molecular weight Mₙ, T_{g,inf} is a glass transition temperature when the molecular weight is infinite, K is a variable related to a free volume of the polymer, and Mₙ is a number average molecular weight of the polymer.

3. The method of claim 2, wherein the polymer structure information is converted into polymer graph data and input into the polymer glass transition temperature property information estimation artificial neural network, and
the polymer graph data includes at least one of:
polymer graph node attachment data that is data representing an inter-attachment relationship between nodes corresponding to respective atoms constituting a repeat unit for composing the polymer and attaching nodes that are attached to an attach point that is a point where the repeat units are repeatedly attached among the nodes;
polymer node attribution information that is data representing attribute information about the nodes corresponding to the respective atoms constituting the repeat unit for composing the polymer and attributes of attaching nodes that are attached to the attach point that is a point where the repeat units are repeatedly attached among the nodes; and
polymer edge attribute information that is information representing attributes of an edge for attaching the nodes corresponding to the respective atoms constituting the repeat unit for composing the polymer to each other and an attach edge for attaching at least one of the nodes to the attaching node.

4. A computer-implemented method for calculating an estimated value of a glass transition temperature of a selected polymer, the computer-implemented method comprising:
generating a machine-trained graph artificial neural network to estimate glass transition temperature property information about the selected polymer based at least in part on a chemical structure related to the selected polymer;
acquiring polymer graph data describing a chemical structure of the selected polymer as graph data;
providing the acquired polymer graph data as input to the graph artificial neural network;
receiving the polymer glass transition temperature property information of the selected polymer from output of the graph artificial neural network; and
calculating a glass transition temperature of the selected polymer by substituting molecular weight information about the selected polymer into the polymer glass transition temperature property information.

5. The computer-implemented method of claim 4, wherein the polymer graph data describing the chemical structure of the selected polymer as graph data includes at least one of:
polymer graph node attachment data that is data representing an inter-attachment relationship between nodes corresponding to respective atoms constituting a repeat unit for composing the selected polymer and attaching nodes that are attached to an attach point that is a point where the repeat units are repeatedly attached among the nodes corresponding to respective atoms constituting a repeat unit for composing the selected polymer;
polymer node attribution information that is data representing attribute information about the nodes corresponding to the respective atoms constituting the repeat unit for composing the selected polymer and attributes of attaching nodes that are attached to the attach point that is a point where the repeat units are repeatedly attached among the nodes corresponding to respective atoms constituting a repeat unit for composing the selected polymer; and
polymer edge attribute information that is information representing attributes of an edge for attaching the nodes corresponding to the respective atoms constituting the repeat unit for composing the selected polymer to each other and an attach edge for attaching at least one of the nodes corresponding to respective atoms constituting a repeat unit for composing the selected polymer to the attaching node.

6. The computer-implemented method of claim 5, wherein providing the acquired polymer graph data as input to the machine-trained graph neural network includes:
constructing a polymer adjacency matrix representing an inter-attachment relationship between two or more nodes representing respective atoms constituting the single molecule; and
inputting the constructed polymer adjacency matrix into the graph artificial neural network, and
the polymer adjacency matrix further comprises attachment relationship information between the attaching node and the nodes corresponding to respective atoms constituting a repeat unit for composing the polymer.

7. The computer-implemented method of claim 4, wherein the graph artificial neural network is trained by:
calculating an estimated value of glass transition temperature property information about a learning polymer from structure information about a plurality of polymers; and
updating neural network parameters to minimize an error between an estimated value of a glass transition temperature calculated by reflecting a measured molecular weight of the corresponding polymer in the calculated estimated value of the glass transition temperature property information and a measured value of the glass transition temperature of the learning polymer.

8. The computer-implemented method of claim 4, wherein generating the machine-trained graph artificial neural network comprises:
acquiring, by one or more computing devices, training data including chemical structures of a plurality of learning polymers and measured values of glass transition temperatures and measured values of molecular weights of the learning polymers having the chemical structures of the learning polymers; and
acquiring graph data graphically describing the chemical structures of the learning polymers and inputting the graph data into the graph neural network, and training the graph neural network so that the estimated values of glass transition temperature property information about the learning polymers output by the graph neural network are output.

9. The computer-implemented method of claim 8, wherein the graph data graphically describing the chemical structures of the learning polymers comprises:
two or more pieces of node data representing respective atoms constituting a single molecule for composing the learning polymer;
one or more pieces of edge data representing coupling between respective atoms;
attaching node data that includes a point where the single molecules are repeatedly attached; and
attach edge data representing attachment between the atom and the attaching node.

10. The computer-implemented method of claim 4, wherein the glass transition temperature property information is a T_{g,inf} value and K in Equation 2:
$${T}_{g} = {T}_{g , inf} - \frac{K}{{M}_{n}} , \qquad [ Equation 2 ]$$
wherein T_{g} is a glass transition temperature of a polymer with a molecular weight Mₙ, T_{g,inf} is a glass transition temperature when the molecular weight is infinite, K is a variable related to a free volume of the polymer, and Mₙ is a number average molecular weight of the polymer.

11. The computer-implemented method of claim 4, wherein
calculating the glass transition temperature of the selected polymer is by substituting number average molecular weight information of the selected polymer into Equation 3:
$${T}_{g} = {T}_{g , inf} - \frac{K}{{M}_{n}} , \qquad [ Equation 3 ]$$
wherein T_{g} is a glass transition temperature of a polymer with a molecular weight Mₙ, T_{g,inf} is a glass transition temperature when the molecular weight is infinite, K is a variable related to a free volume of the polymer, and Mₙ is a number average molecular weight of the polymer.

12. A glass transition temperature calculation system for polymers, comprising:
a data input unit that receives structure information about a selected polymer and molecular weight data of the selected polymer;
an artificial neural network model unit that calculates an estimated value of glass transition temperature property information about the selected polymer from the structure information about the selected polymer; and
a glass transition temperature calculation unit that calculates an estimated value of a glass transition temperature of the selected polymer from the estimated value of the glass transition temperature property information about the selected polymer and the molecular weight data of the selected polymer.

13. The glass transition temperature calculation system for polymers of claim 12, wherein the glass transition temperature property information about the selected polymer is a T_{g,inf} value and a K,
the molecular weight data of the polymer is Mₙ, and
the glass transition temperature calculation unit
calculates a glass transition temperature T_{g} from Equation 4:
$${T}_{g} = {T}_{g , inf} - \frac{K}{{M}_{n}} \qquad [ Equation 4 ]$$
wherein T_{g} is a glass transition temperature of a polymer with a molecular weight Mₙ, T_{g,inf} is a glass transition temperature when the molecular weight is infinite, K is a variable related to a free volume of the polymer, and Mₙ is a number average molecular weight of the polymer.

14. The glass transition temperature calculation system for polymers of claim 13, further comprising a data preprocessing unit that converts the structure information about the selected polymer into selected polymer graph data,
wherein the artificial neural network model unit is machine-trained to calculate the estimated value of the glass transition temperature property information about the selected polymer from the selected polymer graph data, and
the selected polymer graph data includes at least one of:
polymer graph node attachment data that is data representing an inter-attachment relationship between nodes corresponding to respective atoms constituting a repeat unit for composing the selected polymer and attaching nodes that are attached to an attach point that is a point where the repeat units are repeatedly attached among the nodes corresponding to respective atoms constituting a repeat unit for composing the selected polymer;
polymer node attribution information that is data representing attribute information about the nodes corresponding to the respective atoms constituting the repeat unit for composing the selected polymer and attributes of attaching nodes that are attached to the attach point that is a point where the repeat units are repeatedly attached among the nodes corresponding to respective atoms constituting a repeat unit for composing the selected polymer; and
polymer edge attribute information that is information representing attributes of an edge for attaching the nodes corresponding to the respective atoms constituting the repeat unit for composing the selected polymer to each other and an attach edge for attaching at least one of the nodes corresponding to respective atoms constituting a repeat unit for composing the selected polymer to the attaching node.

15. The glass transition temperature calculation system for polymers of claim 12, wherein the artificial neural network model unit includes a polymer glass transition temperature property information estimation artificial neural network configured to receive structure information about a polymer and output an estimated value of the glass transition temperature property information about the polymer,
the polymer glass transition temperature property information estimation artificial neural network, using structure information about a predetermined learning polymer and a measured value of a glass transition temperature and a measured value of a molecular weight of the learning polymer as training data, calculates an estimated value of the glass transition temperature property information about the learning polymer from the structure information about the learning polymer, the polymer glass transition temperature property information estimation artificial neural network being machine-trained by calculating the estimated value of the glass transition temperature of the learning polymer using the estimated value of the glass transition temperature property information and the measure value of the molecular weight and updating neural network parameters of the polymer glass transition temperature property information estimation artificial neural network by comparing the calculated values with the measured value of the glass transition temperature of the learning polymer.

16. The glass transition temperature calculation system for polymers of claim 15, wherein the training data contains data sets including polymer graph data containing structure information about a sample polymer, a measured T_{g} value of the sample polymer, and a molecular weight (Mₙ) of the sample polymer, and
at least a predetermined number of the data sets are data sets of polymers with similar structures and different molecular weights and data sets of polymers with different structures and the same molecular weight.
